(19) United States
(12) Patent Application Publication
(10) Pub. No.: US 2026/0013817 A1
(43) Pub. Date: Jan. 15, 2026

(54) EFFICIENT MEDICAL DATASET PRESENTATION

(71) Applicant: Accuray LLC, Madison, WI (US)

(72) Inventors: Kyle Nethery-Pavelchak, San Jose, CA (US); Richard Holloway, Chapel Hill, NC (US)

(21) Appl. No.: 18/768,822

(22) Filed: Jul. 10, 2024

(51) Int. Cl. A61B 6/00; A61B 8/08; G06T 11/00; G16H 30/40

(52) U.S. Cl. CPC A61B 6/5223; A61B 8/523; G06T 11/008; G16H 30/40; G06T 2210/41

(57) ABSTRACT

A method of remote image data set presentation includes dividing an image viewing space of a client device into a set of three-dimensional (3D) volumes, wherein the image viewing space presents a two-dimensional (2D) viewing plane of data and determining a set of 3D volumes of the image viewing space intersecting the 2D viewing plane. The method further includes retrieving, from a data server, image data associated with each of the 3D volumes intersecting the 2D viewing plane and presenting a portion of the retrieved image data corresponding to a position of the 2D viewing plane within the image viewing space.

### TECHNICAL FIELD

The present disclosure relates to presentation of medical datasets, and more particularly, to efficient retrieval and display of remotely stored datasets.

### BACKGROUND

In networked environments, loading data to a client (e.g., a browser of a client device) requires loading the data over a network connection. The client can cache the retrieved data for navigation once it has been loaded. Medical image datasets can be generated from various sources, such as magnetic resonance imaging (MRI), computed tomography (CT), and so forth. Such medical image datasets can be stored at a data storage server for retrieval by clients. The clients can display the data that has been loaded to the client but the medical image datasets can be large and may cause load time bottlenecks or may overload the cache or memory available to the client. Various access patterns can be employed, depending on the client. Without the full dataset, however, a strategy is needed to isolate and communicate information enough to prepare a request to visualize the data.

### BRIEF DESCRIPTION OF THE DRAWINGS

The present disclosure will be understood more fully from the detailed description given below and from the accompanying drawings of various implementations of the disclosure.

FIG. 1A illustrates a helical radiation delivery system, in accordance with embodiments described herein.

FIG. 1B illustrates a robotic treatment system that may be used in accordance with embodiments described herein.

FIG. 1C illustrates a C-arm gantry-based radiation treatment system, in accordance with embodiments described herein.

FIG. 2 illustrates an example system for remote data set presentation, in accordance with embodiments described herein.

FIG. 3A illustrates an example image viewing space and image viewing plane, in accordance with embodiments described herein.

FIG. 3B illustrates another example image viewing space and image viewing plane, in accordance with embodiments described herein.

FIG. 3C illustrates an example 2D viewing plane, in accordance with embodiments described herein.

FIG. 4 illustrates an example of a mapping between volumes in an image viewing space and volumes of image data, in accordance with embodiments described herein.

FIG. 5 illustrates an example of datasets and timeseries of datasets, in accordance with embodiments described herein.

FIG. 6 depicts a flow chart illustrating an example method of remote data set presentation, in accordance with embodiments described herein.

FIG. 7 depicts a flow chart illustrating an example method of fast and intelligent presentation of remote medical data sets, in accordance with embodiments described herein.

FIG. 8 is a block diagram of an example apparatus that may perform one or more of the operations described herein, in accordance with some embodiments of the present disclosure.

### DETAILED DESCRIPTION

As discussed above, in networked environments, loading data to a client device requires retrieving (e.g., loading/transmitting) the data over a network connection from a data server storing the data. The client device may cache data (e.g., in local memory) for rapid navigation once the data has been loaded. However, the load times for large medical datasets can be a bottleneck, causing long load times and hindering the ability of the user to navigate through the dataset effectively. Additionally, loading multiple datasets may overload the cache on the client which may cause the load to fail or for previously retrieved data to be overwritten. For medical applications such as radiotherapy, the client device may be, for example, a radiation oncologist's laptop and the server may be a picture archiving and communication system (PACS) or other system that holds the patient images. If loaded directly into memory of the client, for example, a 1024³ image consumes 1 gigabyte of memory space. Similarly, medical datasets that include 16 bit values of type ‘short’ may consume 2 gigabytes of memory space (e.g., per Digital Imaging and Communications in Medicine (DICOM) standards). When overlaying multiple images, space requirements increase linearly. A routine use case in radiotherapy includes physician or physicist review of plan information. Such users may not be in the clinic when the need arises for image data review. Accordingly, a responsive client-server system for viewing and interacting with images and related data may benefit the image data review process.

Described herein are embodiments of systems and methods to organize and efficiently present datasets (e.g., radiotherapy, medical, or other imaging datasets) over a network, such as via a client-server connection. In some embodiments, low-fidelity data may be retrieved and displayed by a client device from a server while full-resolution data is retrieved. Additionally, in some embodiments, a smart caching system may pre-fetch and cache full-resolution data that is likely to be viewed, such as image data that is near a currently viewed plane or of critical data that is likely to be viewed in the normal course of the workflow. For example, an image viewing space of the client device may be divided into a set of volumes. Additionally, the image data stored at the server may also be divided into similar sized volumes. As a viewing plane of the client device (e.g., the 2D slice of data that can be displayed) is adjusted or moved (e.g., positionally or temporally), the data corresponding to the volumes of the image viewing space that are intersected by the viewing plane may be retrieved and cached. Thus, only a subsection of the high-resolution image data is fetched based on the position of the viewing plane and the probability of data being viewed. As referred to herein, position of the viewing plane may include a translational position and orientation of the viewing plane within the 3D viewing space.

Advantageously, the embodiments described herein provide near-instant client load of large remote datasets for review. Because data is fetched intelligently on an as-needed basis, only a portion of the entire subset may be loaded at any given time to allow reduced load time and minimizing the loading of unnecessary data that is not viewed. Accordingly, memory usage of the client device may be reduced and the image data may be displayed with little to no perceivable delay. Additionally, because low-fidelity data is displayed first while high-resolution data is obtained, any updating of the displayed image data may be performed with minimal delay or in real-time with the low-resolution data until the high-resolution data is retrieved and presents, thus providing seamless user interaction and viewing of the entire volume of image data.

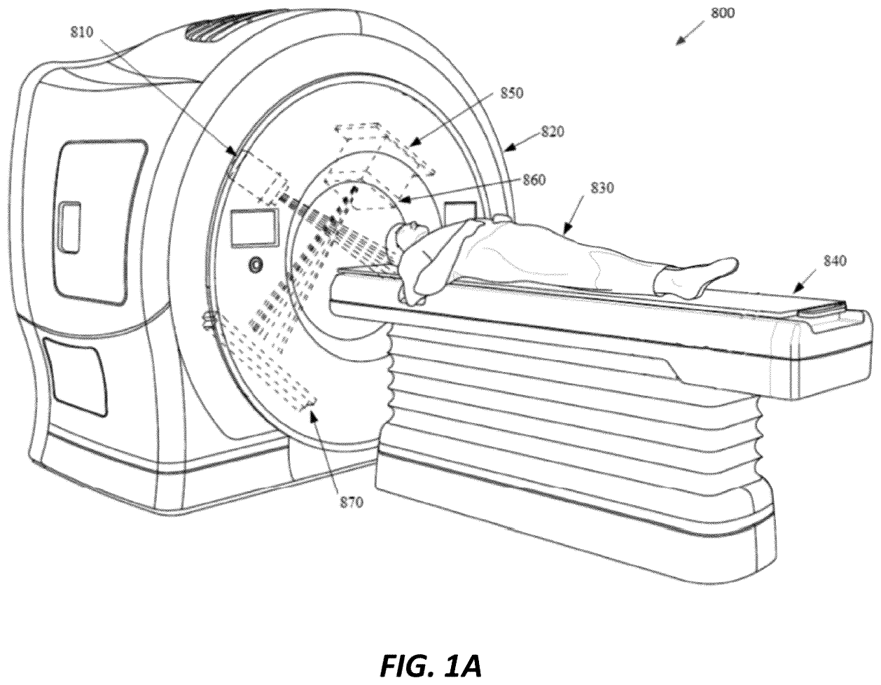

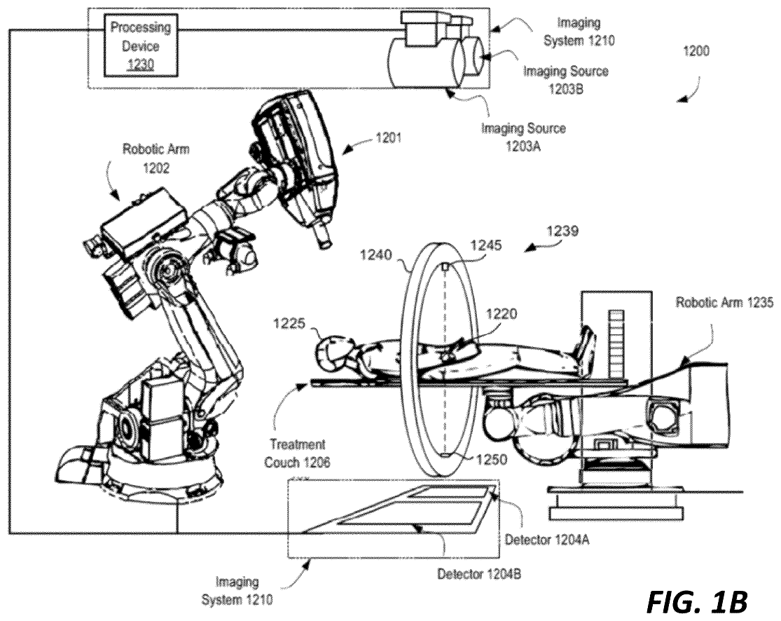

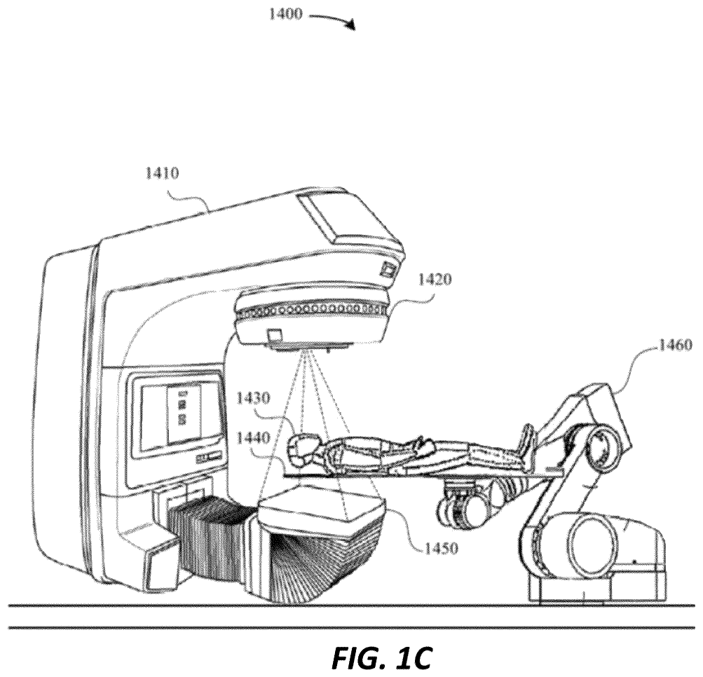

FIG. 1A illustrates a helical radiation delivery system 800 in accordance with embodiments of the present disclosure. The helical radiation delivery system 800 may include a linear accelerator (LINAC) 850 mounted to a ring gantry 820. The LINAC 850 may be used to generate a radiation beam (i.e., treatment beam) by directing an electron beam towards an x-ray emitting target. The treatment beam may deliver radiation to a target region (e.g., a tumor). The treatment system further includes a multi-leaf collimator (MLC) 860. The MLC includes a housing that houses multiple leaves that are movable to adjust an aperture of the MLC to enable shaping of the treatment beam. The ring gantry 820 has a toroidal shape in which the patient 830 extends through a bore of the ring/toroid and the LINAC 850 is mounted on the perimeter of the ring and rotates about the axis passing through the center to irradiate a target region with beams delivered from one or more angles around the patient. During treatment, the patient 830 may be simultaneously moved through the bore of the gantry on a treatment couch 840.

The helical radiation delivery system 800 includes an imaging system, comprising the LINAC 850 as an imaging source and an x-ray detector 870. The LINAC 850 may be used to generate a mega-voltage x-ray image (MVCT) or a kilo-voltage x-ray image (kVCT) of a region of interest (ROI) of patient 830 by directing a sequence of x-ray beams at the ROI which are incident on the x-ray detector 870 opposite the LINAC 850 to image the patient 830 for setup and generate pre-treatment images. In one embodiment, the helical radiation delivery system 800 may also include a secondary imaging system consisting of a kV imaging source 810 mounted orthogonally relative to the LINAC 850 (e.g., separated by 90 degrees) on the ring gantry 820 and may be aligned to project an imaging x-ray beam at a target region and to illuminate an imaging plane of a detector after passing through the patient 130.

FIG. 1B illustrates a radiation treatment system 1200 that may be used in accordance with alternative embodiments described herein. As shown, FIG. 1B illustrates a configuration of a radiation treatment system 1200. In the illustrated embodiments, the radiation treatment system 1200 includes a linear accelerator (LINAC) 1201 that acts as a radiation treatment source and an MLC 1205 in mounted in front of the LINAC to shape the treatment beam. In one embodiment, the LINAC 1201 is mounted on the end of a robotic arm 1202 having multiple (e.g., 5 or more) degrees of freedom in order to position the LINAC 1201 to irradiate a target (e.g., pathological anatomy such as a tumor or other anatomy that may benefit from irradiation such as an acoustic neuroma) with beams delivered from many angles, in many planes, in an operating volume around a patient. Treatment may involve beam paths with a single isocenter, multiple isocenters, or with a non-isocentric approach.

LINAC 1201 may be positioned at multiple different nodes (predefined positions at which the LINAC 1201 is stopped and radiation may be delivered) during treatment by moving the robotic arm 1202. At the nodes, the LINAC 1201 can deliver one or more radiation treatment beams to a target, where the radiation beam shape is determined by the leaf positions in the MLC 1205. The nodes may be arranged in an approximately spherical distribution about a patient. The particular number of nodes and the number of treatment beams applied at each node may vary as a function of the location and type of pathological anatomy to be treated.

The radiation treatment system 1200 includes an imaging system 1210 having a processing device 1230 connected with x-ray sources 1203A and 1203B (i.e., imaging sources) and fixed x-ray detectors 1204A and 1204B. Alternatively, the x-ray sources 1203A, 1203B and/or x-ray detectors 1204A, 1204B may be mobile, in which case they may be repositioned to maintain alignment with the target, or alternatively to image the target from different orientations or to acquire many x-ray images and reconstruct a three-dimensional (3D) cone-beam CT. In one embodiment, the x-ray sources are not point sources, but rather x-ray source arrays, as would be appreciated by the skilled artisan. In one embodiment, LINAC 1201 serves as an imaging source, where the LINAC power level is reduced to acceptable levels for imaging.

Imaging system 1210 may perform computed tomography (CT) such as cone beam CT or helical megavoltage computed tomography (MVCT), and images generated by imaging system 1210 may be two-dimensional (2D) or three-dimensional (3D). The two x-ray sources 1203A and 1203B may be mounted in fixed positions on the ceiling of an operating room and may be aligned to project x-ray imaging beams from two different angular positions (e.g., separated by 90 degrees) to intersect at a machine isocenter (referred to herein as a treatment center, which provides a reference point for positioning the patient on a treatment couch 1206 during treatment) and to illuminate imaging planes of respective detectors 1204A and 1204B after passing through the patient. In one embodiment, imaging system 1210 provides stereoscopic imaging of a target and the surrounding volume of interest (VOI). In other embodiments, imaging system 1210 may include more or less than two x-ray sources and more or less than two detectors, and any of the detectors may be movable rather than fixed. In yet other embodiments, the positions of the x-ray sources and the detectors may be interchanged. Detectors 1204A and 1204B may be fabricated from a scintillating material that converts the x-rays to visible light (e.g., amorphous silicon), and an array of CMOS (complementary metal oxide silicon) or CCD (charge-coupled device) imaging cells that convert the light to a digital image that can be compared with a reference image during an image registration process that transforms a coordinate system of the digital image to a coordinate system of the reference image, as is well known to the skilled artisan. The reference image may be, for example, a digitally reconstructed radiograph (DRR), which is a virtual x-ray image that is generated from a 3D CT image based on simulating the x-ray image formation process by casting rays through the CT image.

In one embodiment, IGRT delivery system 1200 also includes a secondary imaging system 1239. Imaging system 1239 is a Cone Beam Computed Tomography (CBCT) imaging system, for example, the medPhoton ImagingRing System. Alternatively, other types of volumetric imaging systems may be used. The secondary imaging system 1239 includes a rotatable gantry 1240 (e.g., a ring) attached to an arm and rail system (not shown) that move the rotatable gantry 1240 along one or more axes (e.g., along an axis that extends from a head to a foot of the treatment couch 1206. An imaging source 1245 and a detector 1250 are mounted to the rotatable gantry 1240. The rotatable gantry 1240 may rotate 360 degrees about the axis that extends from the head to the foot of the treatment couch. Accordingly, the imaging source 1245 and detector 1250 may be positioned at numerous different angles. In one embodiment, the imaging source 1245 is an x-ray source and the detector 1250 is an x-ray detector. In one embodiment, the secondary imaging system 1239 includes two rings that are separately rotatable. The imaging source 1245 may be mounted to a first ring and the detector 1250 may be mounted to a second ring. In one embodiment, the rotatable gantry 1240 rests at a foot of the treatment couch during radiation treatment delivery to avoid collisions with the robotic arm 1202.

As shown in FIG. 1B, the image-guided radiation treatment system 1200 may further be associated with a treatment delivery workstation 150. The treatment delivery workstation may be remotely located from the radiation treatment system 1200 in a different room than the treatment room in which the radiation treatment system 1200 and patient are located. The treatment delivery workstation 150 may include a processing device (which may be processing device 1230 or another processing device) and memory that modify a treatment delivery to the patient 1225 based on a detection of a target motion that is based on one or more image registrations, as described herein.

FIG. 1C. Illustrates a C-arm radiation delivery system 1400. In one embodiment, in the C-arm system 1400 the beam energy of a LINAC may be adjusted during treatment and may allow the LINAC to be used for both x-ray imaging and radiation treatment. In another embodiment, the system 1400 may include an onboard kV imaging system to generate x-ray images and a separate LINAC to generate the higher energy therapeutic radiation beams. The system 1400 includes a gantry 1410, a LINAC 1420, an MLC 1470 in front of the LINAC 1420 to shape the beam, and a portal imaging detector 1450. The gantry 1410 may be rotated to an angle corresponding to a selected projection and used to acquire an x-ray image of a VOI of a patient 1430 on a treatment couch 1440. In embodiments that include a portal imaging system, the LINAC 1420 may generate an x-ray beam that passes through the target of the patient 1430 and are incident on the portal imaging detector 1450, creating an x-ray image of the target. After the x-ray image of the target has been generated, the beam energy of the LINAC 1420 may be increased so the LINAC 1420 may generate a radiation beam to treat a target region of the patient 1430. In another embodiment, the kV imaging system may generate an x-ray beam that passes through the target of the patient 1430, creating an x-ray image of the target. In some embodiments, the portal imaging system may acquire portal images during the delivery of a treatment. The portal imaging detector 1450 may measure the exit radiation fluence after the beam passes through the patient 1430. This may enable internal or external fiducials or pieces of anatomy (e.g., a tumor or bone) to be localized within the portal images.

Alternatively, the kV imaging source or portal imager and methods of operations described herein may be used with yet other types of gantry-based systems. In some gantry-based systems, the gantry rotates the kV imaging source and LINAC around an axis passing through the isocenter. Gantry-based systems include ring gantries having generally toroidal shapes in which the patient's body extends through the bore of the ring/toroid, and the kV imaging source and LINAC are mounted on the perimeter of the ring and rotates about the axis passing through the isocenter. Gantry-based systems may further include C-arm gantries, in which the kV imaging source and LINAC are mounted, in a cantilever-like manner, over and rotates about the axis passing through the isocenter. In another embodiment, the kV imaging source and LINAC may be used in a robotic arm-based system, which includes a robotic arm to which the kV imaging source and LINAC are mounted as discussed above. Aspects of the present disclosure may further be used in other such systems such as a gantry-based LINAC system, static imaging systems associated with radiation therapy and radiosurgery, proton therapy systems using an integrated image guidance, interventional radiology and intraoperative x-ray imaging systems, etc.

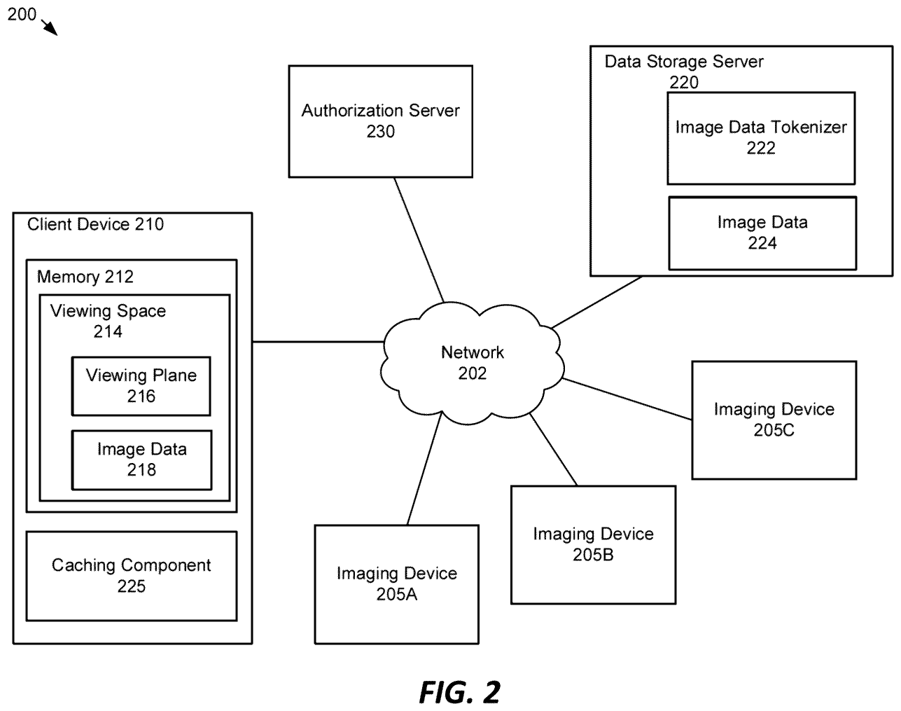

FIG. 2 illustrates an example system 200 for remote data set presentation, in accordance with embodiments described herein. System 200 includes a client device 210 coupled with a data storage server 220 and an authorization server 230 via a network 202. Additionally, the data storage server 220 may be coupled with imaging devices 205A-C(e.g., such as imaging devices described with respect to FIGS. 1A-B) via the network 202. In some embodiments, the data storage server 220 may be any computing device operable to communicate via a network, such as a desktop computer, a laptop computer, a mainframe computer, a personal digital assistant, a rack-mount server, a hand-held device or any other device configured to process data. The data storage server 220 may store image data 224 (e.g., captured by one or more of imaging devices 205A-C). The image data 224 may include any digitally stored data generated via an imaging system or technique, such as magnetic resonance imaging (MRI), computed tomography (CT), positron emission tomography (PET), ultrasound, or any other imaging technique. In some embodiments, the data storage server 220 may include logic (e.g., image data tokenizer 222), to tokenize or divide the image data 224 into sub-volumes. For example, the collection of image data 224 may represent a three-dimensional space and the image data tokenizer 222 may divide that three-dimensional space into several or any number of sub-volumes (e.g., a geometrical volume such as a rectangular or square cubic volume), each representing a portion of the 3-D image data 224. Each sub-volume may be represented or identified with a coordinate defining the sub-volume location within the 3-D image data 224.

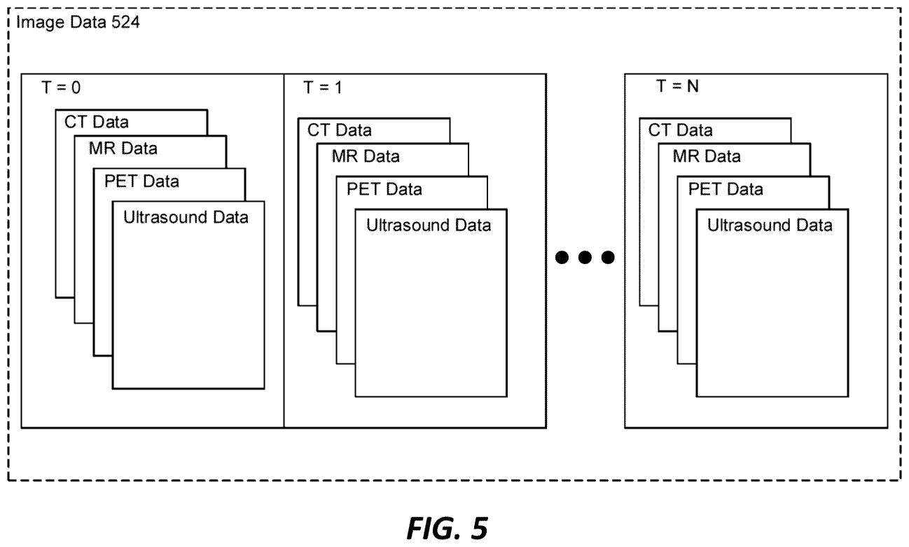

In some embodiments, the client device 210 may be a any data processing device, such as a desktop computer, a laptop computer, a mainframe computer, a personal digital assistant, a rack-mount server, a hand-held device or any other device configured to process data. The client device 210 may include a viewing space 214 defined within a memory 212 of the client device 210 to receive and cache image data 218. The viewing space 214 may defined a 3-D coordinate system in which image data 218 may be cached and displayed. In some embodiments, the viewing space 214 may include a viewing plane 216 (e.g., a camera plane) that defines a two-dimensional cross-section of image data 218 within the viewing space 214 that is to be rendered and displayed by a graphical user interface of the client device 210. For example, when a user views the image data 218 within the viewing space 214, the user may move the viewing plane 216 in three-dimensions (e.g., via translation, rotation, etc.). In some embodiments, the user may also move the viewing plane 216 with respect to time, in addition to movement within the 3-D viewing space 214, as described in more detail with respect to FIG. 5 below. Accordingly, the 2-D viewing plane 216 may define the image data displayed to the user depending on the location and time coordinates of the viewing plane 216 within the viewing space 214.

In some embodiments, the viewing space 214 may be divided into multiple sub-volumes that correspond and map to the sub-volumes of the image data 224 defined by the image data tokenizer 222. For example, the coordinates for each sub-volume of the image data 224 may correspond to the same coordinates for the sub-volumes defined within the viewing space 214. Thus, when a sub-volume of the image data 224 is loaded from the data storage server 220 to the client device 210, the sub-volume of image data 224 can be loaded into the corresponding location in the viewing space 214 as image data 218.

In some embodiments, the client device 210 may include a caching component 225 to determine which data (e.g., which sub-volumes of data) to retrieve from the data storage server 220. For example, as the viewing plane 216 is initialized or as a user moves the viewing plane 216 within the viewing space 214, the caching component 225 may determine which sub-volumes of the viewing space 214 that the viewing plane 216 intersects. The caching component 225 may then determine if the image data 218 for those intersected volumes has been cached in memory 212. If the data is not cached for the intersected volumes (e.g., a cache miss occurs), the caching component 225 may retrieve image data 224 for those corresponding volumes (e.g., based on a coordinate of the intersected volumes). In some embodiments, the caching component 225 may first retrieve low-resolution or highly compressed data for the intersected volumes to reduce render time. The caching component 225 may then retrieve high resolution or full data to replace the low-resolution or highly compressed data.

In some embodiments, the caching component 225 may further identify or predict which volumes of data are likely to be viewed by the user and pre-emptively retrieve such data in anticipation of the navigation by the user. For example, to maximize image fidelity and responsiveness, the caching component may identify high priority data based on various factors. In some embodiments, such factors may include a tumor location or distance from tumor location, organs at risk, dose (e.g., prioritizing high dose regions), a score card for planning goals or constraints, density, user interaction (e.g., direction of scroll or movement of the viewing plane 216), etc. Additionally, in some embodiments an artificial intelligence model may be trained to predict likely movements of the viewing plane 216 based on previous user sessions and navigation patterns, similar plan parameters, and so forth. Accordingly, data for sub-volumes that are likely to be viewed can be retrieved proactively to provide a faster, more efficient loading of large image datasets thus enabling highly responsive image rendering and viewing at the client device 210.

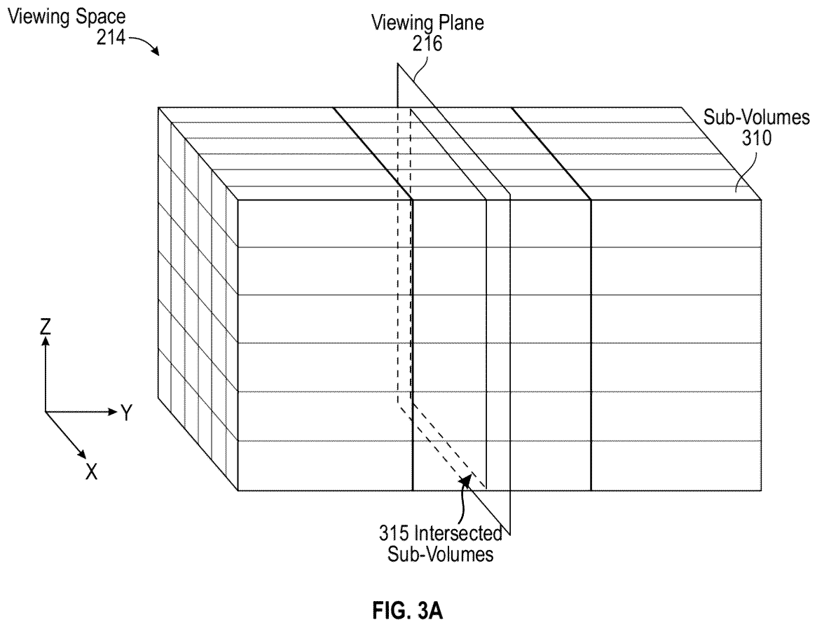

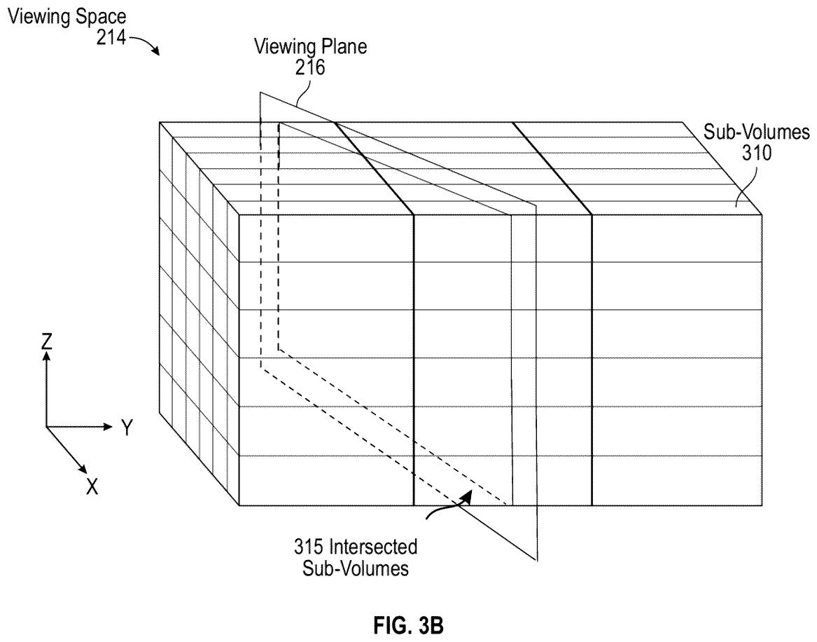

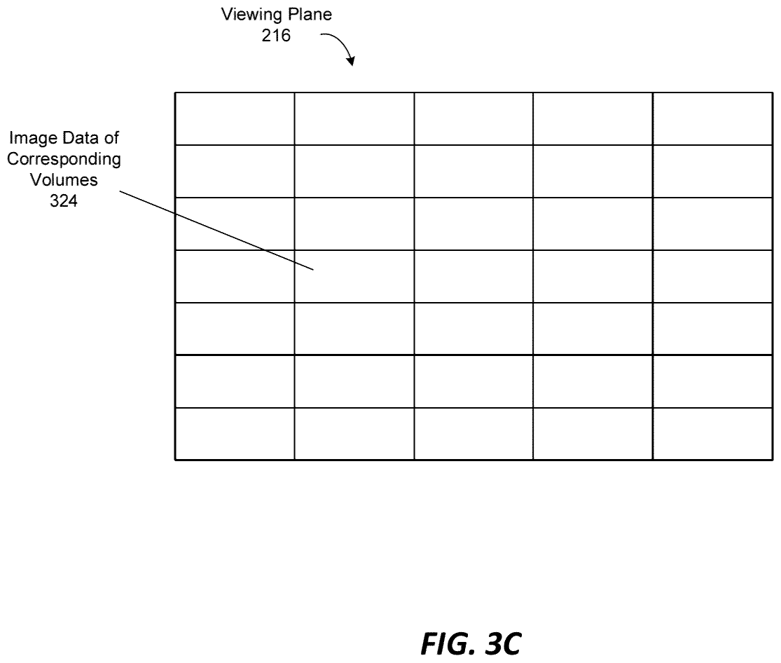

FIG. 3A illustrates an example image viewing space and image viewing plane, in accordance with embodiments described herein. As depicted, the viewing space 214 may include several sub-volumes 310 that are aligned in a coordinate system to represent a 3-D space. Each of the sub-volumes 310 may represent and correspond to a sub-volume of image data stored at a data storage server. For example, the coordinate system of the viewing space 214 may be normalized with a 3-D space represented within a set of image data. For example, one or more 3-D scans may be performed on a patient, the data from which may be stored at a data storage server. The data from the 3-D scans may be divided, or tokenized, into several sub-volumes that correspond to the sub-volumes 310 of the viewing space. The viewing plane 216 may represent a camera plane or view of the image data. For example, the viewing plane 216 is the plane of data that is presented and viewable by a user (e.g., via a user interface of a client device). In some embodiments, processing logic may determine which of the sub-volumes 310 of the viewing space 214 are being intersected by the viewing plane 216. For example, as depicted in FIG. 3A, the viewing plane 216 intersects each of the central column sub-volumes 310 (e.g., intersected sub-volumes 315). The intersected sub-volumes 315 may each include or be associated with a set of coordinates representing the position of each intersected sub-volume 315 within the coordinate system of the viewing space 214. Because the coordinate system of the viewing space 214 and the coordinate system of the divided image data directly correspond (e.g., are mapped 1-to-1), the respective image data of the intersected sub-volumes 315 may be identified and retrieved from the image data stored at the data storage server. The retrieved data may be for the entire volume, rather than just for the viewing plane 216. Accordingly, the processing logic may then determine the particular data that corresponds to the position of the viewing plane 216 and display the data via user interface. As discussed above, the processing logic may first retrieve low-resolution data which may load quickly to provide an initial display, after which high resolution data may be retrieved to provide a better image quality. Thus, the low-resolution data may load more quickly for ease of navigation while the high-resolution data may provide high detail for review and analysis of regions of interest.

FIG. 3B illustrates another example image viewing space and image viewing plane, in accordance with embodiments described herein. As depicted in FIG. 3B, the viewing plane 216 may be translated and rotated within the coordinate system of the viewing space 214. Thus, various different sub-volumes 310 may be intersected by the viewing plane 216 during the translation or rotation, triggering the retrieval of additional image data corresponding to the newly intersected sub-volumes 320.

FIG. 3C illustrates an example 2D viewing plane 216, in accordance with embodiments described herein. As described above with respect to FIGS. 3A and 3B, the viewing plane 216 may intersect with various sub-volumes 310 within an image viewing space 214. The image data for each of the intersected sub-volumes are then retrieved from a remote data storage server. The data lying along the viewing plane 216 may then be displayed via a graphical user interface. FIG. 3C depicts an example cross-section of the intersected sub-volumes of the viewing space 214. As can be seen, many sub-volumes may be intersected by the viewing plane 216, each of which may trigger the retrieval of corresponding image data. The image data lying along the viewing plane 216 may then be rendered and displayed (e.g., displaying data from the image data of each of the corresponding volumes 324). Accordingly, a displayed image may be reproduced from the data retrieved for the intersected sub-volumes. For simplicity of depiction and description, FIG. 3C illustrates an example in which the viewing plane 216 intersects orthogonally across the rectangular sub-volumes of the viewing space. Accordingly, the data displayed from each sub-volume (e.g., the portions of the sub-volumes intersected by the viewing plane 216) may take many different shapes, such as when the viewing plane 216 intersects the sub-volumes at an oblique angle. According, the tile identification is considered as the intersection of a rectangular, three-dimensional, or volumetric dataset and a plane (e.g., the viewing plane 216), which can be visualized as various different shapes (e.g., triangle, rectangle, square, etc.) depending on the angle of intersection,

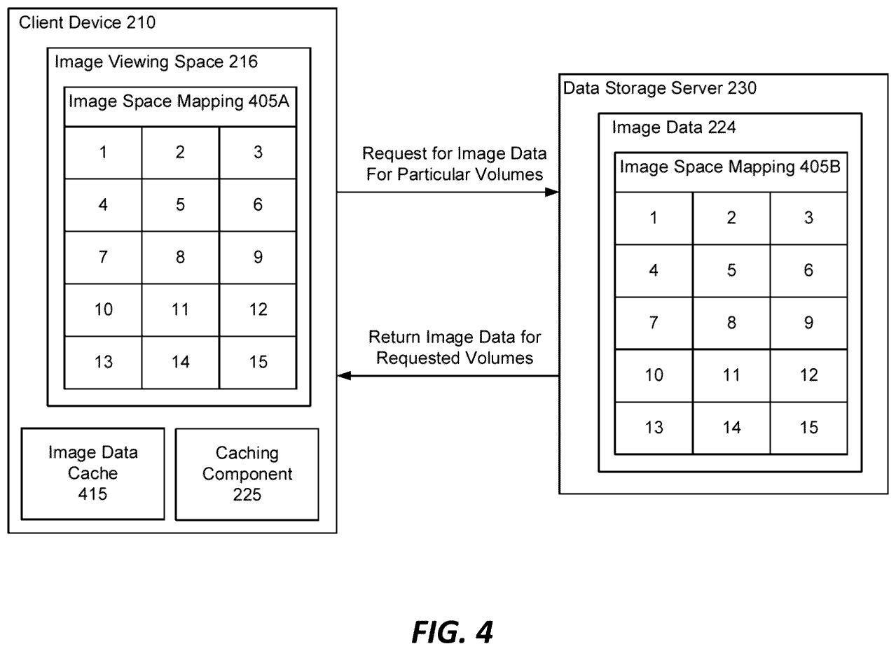

FIG. 4 illustrates an example of a mapping between volumes in an image viewing space and volumes of image data, in accordance with embodiments described herein. In some embodiments, a client device 210 may include an image viewing space 216 including an image space mapping 405A of several sub-volumes of a 3-D space (e.g., defined by coordinates of the image viewing space 216). Additionally, a data storage server 230 may include image data 224 which may be divided or tokenized into volumes that correspond to the sub-volumes defined by the image viewing space 216. For example, the image viewing space 216 and the image data 224 may each define a 3-D space of a same or similar size, each of which are subdivided into several sub-volumes (e.g., the same number and size of sub-volumes). In some embodiments, the image viewing space 216 may define an abstract space in which image data may be rendered while the image data 224 defines a physical space represented by the 3-D image data. Thus, the sub-volumes of the viewing space 216 may be defined to correspond to a certain physical volume depicted by the image data 224. Therefore, each sub-volume may be mapped one-to-one in the image viewing space 216 and the tokenized image data 224. For example, as depicted, block 1 of the image space mapping 405A may correspond to block 1 of the image space mapping 405B of the image data 224. Therefore, as discussed above, processing logic such as caching component 225 may identify which blocks (e.g., volumes) of the image viewing space are intersected by an image viewing plane. The caching component 225 may determine an identifier or coordinate of each of the intersected volumes. For example, the caching component may determine that blocks 1, 2, 5, 6 and 9 are intersected by the viewing plane in the image viewing space 216, as discussed with respect to FIGS. 3A-C. The client device (e.g., view the caching component 225) may request image data for those particular corresponding volumes (e.g., blocks 1, 2, 5, 6, and 9). The data storage server 230 may then respond to the request by providing the image data from blocks 1, 2, 5, 6, and 9, defined by the image space mapping 405B. The caching component 225 may then cache (e.g., in image data cache 415) the retrieved image data for those blocks. Thus, whenever the viewing plane moves within those retrieved volumes, the cached data in image data cache 415 may be retrieved locally. Additional image data may be retrieved when additional or new volumes are intersected by the viewing plane.

FIG. 5 illustrates an example of datasets and timeseries of datasets, in accordance with embodiments described herein. As mentioned above, a user may translate the viewing plane not only in space (e.g., positional translation) but also in time (e.g., temporal translation) as well as modality or type of image data (e.g., selecting from CT, PET, MRI, etc. or a combination of such). In other words, the image data may include sequences of 3-D scans or images that depict multiple 3-D images over a period of time. For example, image data 524 which may be stored at a data storage server, may include N sets of 3-D scans or images. In some examples, at each time T, the data set may include CT data, MR data, PET data, ultrasound data and/or any other image data that may be collected. In some embodiments, the data set may include any combination or the CT data, MR data, PET data, ultrasound data, etc. Similarly, multiples of the same type of datasets may be included in the data sets (e.g., multiple CT or MR data). Accordingly, any number or combination of types or modalities of data may exist in the data set. In some embodiments, when the image data is retrieved for a sub-volume of the viewing space, as described above, each set of image data may be retrieved including all different temporal sets as well. Alternatively, when image data is retrieved a set of temporal datasets may be retrieved within a certain time period of the temporal coordinate defined by the viewing plane. For example, the user may be viewing a set of image data at time T=5 and data sets that are within two temporal units are retrieved, for example. Therefore, all data for the sub-volumes intersected by the viewing plane would be retrieved for T=3 to T=7. Thus, a range of temporal data may be retrieved based on the likelihood of those data being viewed.

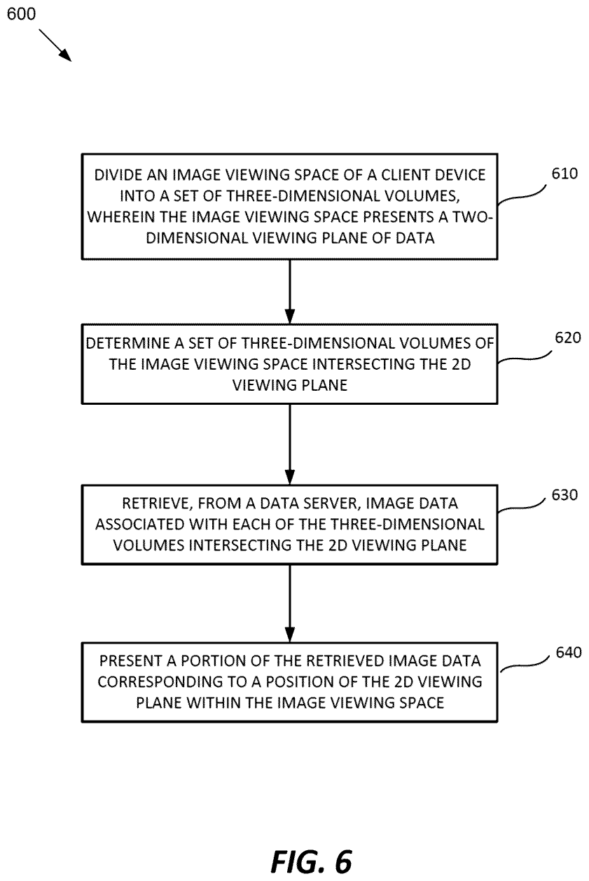

FIG. 6 illustrates an example method of remote data set presentation, in accordance with embodiments described herein. Method 600 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, at least a portion of method 600 may be performed by caching component 225 of FIG. 2.

With reference to FIG. 6, method 600 illustrates example functions used by various embodiments. Although specific function blocks (“blocks”) are disclosed in method 600, such blocks are examples. That is, embodiments are well suited to performing various other blocks or variations of the blocks recited in method 600. It is appreciated that the blocks in method 600 may be performed in an order different than presented, and that not all of the blocks in method 600 may be performed.

Method 600 begins at block 610, where processing logic divides an image viewing space of a client device into a set of three-dimensional (3D) volumes, wherein the image viewing space presents a two-dimensional (2D) viewing plane of data. Alternatively, in some embodiments, the image viewing space presents a 3D viewing volume in which data retrieved may be rendered and displayed in 3D.

At block 620, the processing logic determines a set of 3D volumes of the image viewing space intersecting the 2D viewing plane. In some embodiments, the processing logic may also determine a second set of 3D volumes of the image viewing space corresponding to image data with a high probability of being viewed. For example, image data that corresponds to a region of interest, such as a tumor, may be indicated as having a high probability of being viewed and may be preemptively retrieved.

At block 630, the processing logic retrieves, from a data server, image data associated with each of the 3D volumes intersecting the 2D viewing plane. In some embodiments, each 3D volume of the image viewing space is mapped to image data corresponding to a same position of the 3D volume within the image viewing space. In some embodiments, the image data includes time series data of a plurality of images collected over time. In some embodiments, the image data includes one or more of computed tomography (CT) data, magnetic resonance (MR) data, positron emission tomography (PET) data, and ultrasound data.

At block 640, the processing logic presents a portion of the retrieved image data corresponding to a position of the 2D viewing plane within the image viewing space. In some embodiments, presenting the portion of the retrieved image data includes presenting a combination of two or more of the CT data, MR data, PET data, and ultrasound data. As discussed above, in some embodiments, rather than a 2D viewing plane, the processing logic may present a portion of the retrieved image data corresponding to a position of a 3D viewing volume within the image viewing space.

In some embodiments, the processing logic displays a first subset of the time series data corresponding to a first time and the position of the 2D viewing pane within the image viewing space. The processing logic may then receive an indication of a second time and display a second subset of the time series data of the plurality of images corresponding to the second time and the position of the 2D viewing plane within the image viewing space. In other words, the processing logic may receive a translation of the 2D viewing plane through time and retrieve or display the time series data corresponding to the selected time.

In some embodiments, processing logic may retrieve lower-resolution image data (e.g., 64\hat{ }3 pixel image) for the 3D volumes, initiate retrieval of the higher-resolution image data (e.g., 1024\hat{ }3 pixel image) for the 3D volumes, and display the lower-resolution image data until the higher-resolution image data has been retrieved. In some embodiments, processing logic may receive a second position of the 2D viewing plane within the image viewing space, determine a second set of 3D volumes intersecting the 2D viewing plane at the second position, retrieve image data corresponding to the second set of 3D volumes, and display a portion of the image data for the second position of the 2D viewing plane. In other words, the processing logic may receive a translation or rotation of the 2D viewing plane within the image viewing space and determine if any additional volumes of the viewing space are intersected after which the image data for those volumes is retrieved for display.

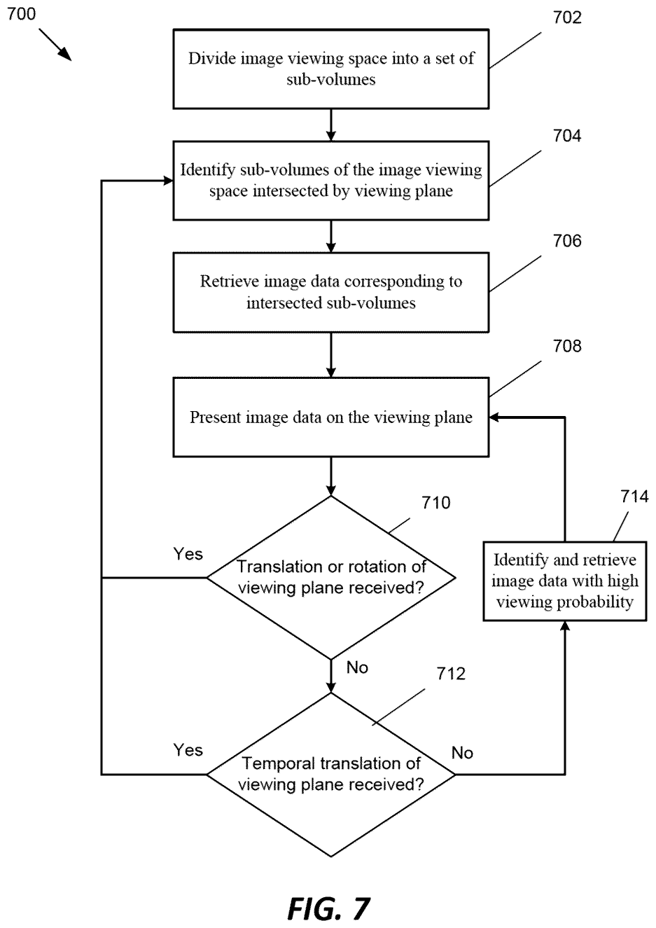

FIG. 7 depicts a flow chart illustrating an example method of fast and intelligent presentation of remote medical data sets, in accordance with embodiments described herein. Method 700 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, at least a portion of method 700 may be performed by caching component 225 of FIG. 2.

With reference to FIG. 7, method 700 illustrates example functions used by various embodiments. Although specific function blocks (“blocks”) are disclosed in method 700, such blocks are examples. That is, embodiments are well suited to performing various other blocks or variations of the blocks recited in method 700. It is appreciated that the blocks in method 700 may be performed in an order different than presented, and that not all of the blocks in method 700 may be performed.

Method 700 begins at block 702, where processing logic divides an image viewing space of a client device into a set of sub-volumes. For example, the sub-volumes may be geometric volumes, such as cubes or rectangular tiles, which may represent a portion of a 3D space. At block 704, processing logic identifies sub-volumes of the image viewing space that are intersected by a viewing plane (e.g., a camera plane for displaying a 2D plane of data to a user).

At block 706, processing logic retrieves image data corresponding to the intersected sub-volumes. For example, the image data may be divided into similar 3D volumes as the image viewing space. Therefore, a mapping such as nominal coordinates of the intersected volumes may be used to identify the corresponding image data stored at the data storage server. At block 708, processing logic may present the image data that lies on the viewing plane (e.g., via a graphical user interface).

At block 710, processing logic determines whether a translation or rotation of the viewing plane within the viewing space has been received. If a translation or rotation of the viewing plane has been received then the process may return to block 704 to identify the sub-volumes of the image viewing space that are intersected by the new position of the viewing plane. Otherwise, if no positional translation or rotation of the viewing plane has been received, the process may proceed to block 712, where processing logic determines if a temporal translation of the viewing plane has been received. Again, if a temporal translation of the viewing plane has been received, the process may return to block 704 to identify the sub-volumes and the temporal data to be retrieved. In some embodiment, all temporal data is retrieved upon the first intersection or caching of the image data for a sub-volume in which case the process would return to block 708 to present the image data corresponding to the temporal translation.

At block 714, if no translations have been received then the processing logic may identify and retrieve image data with high viewing probability to be cached at the client device for quick access. For example, the processing logic may identify sub-volumes of the viewing space that have a high likelihood of being viewed based on various factors and data collected about user navigation. In some embodiments, processing logic may identify sub-volumes including regions of interest such as tumors, at risk organs, etc. or any other items that may necessitate review by a physician reviewing the data, as high probability and initiate preemptive retrieval of such volumes. In some embodiments, the processing logic may identify sub-volumes that are local to or near a current position of the viewing plane as high probability and preemptively retrieve such data once the immediately required image data (e.g., the presently intersected volumes) has already been retrieved (e.g., during client idle time). Similarly, in some embodiments, processing logic may apply an artificial intelligence model to the image data and user navigation to predict which sub-volumes the user is likely to navigate to. For example, the artificial intelligence model may be trained using historical image data sets and past navigation patterns of various users and/or the present user. Accordingly, the artificial intelligence model may identify expected navigation patterns of the user to determine which sub-volumes are likely to be viewed. The processing logic may then preemptively retrieve the corresponding image data for those sub-volumes determined as likely to be viewed.

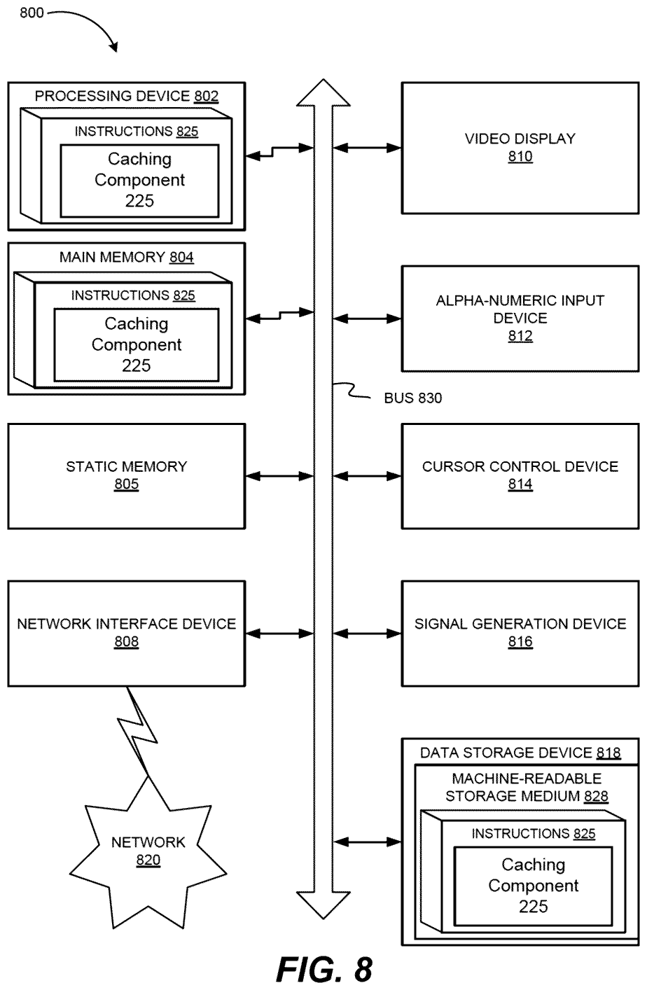

FIG. 8 is a block diagram of an example computing device 800 that may perform one or more of the operations described herein, in accordance with some embodiments. Computing device 800 may be connected to other computing devices in a LAN, an intranet, an extranet, and/or the Internet. The computing device may operate in the capacity of a server machine in client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein.

The example computing device 800 may include a processing device (e.g., a general purpose processor, a PLD, etc.) 802, a main memory 804 (e.g., synchronous dynamic random access memory (DRAM), read-only memory (ROM)), a static memory 806 (e.g., flash memory and a data storage device 818), which may communicate with each other via a bus 830.

Processing device 802 may be provided by one or more general-purpose processing devices such as a microprocessor, central processing unit, or the like. In an illustrative example, processing device 802 may comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 802 may also comprise one or more special-purpose processing devices such as an application specific integrated circuit (ASIC), a field programmable gate array (FPGA), a digital signal processor (DSP), network processor, or the like. The processing device 802 may be configured to execute the operations described herein, in accordance with one or more aspects of the present disclosure, for performing the operations and steps discussed herein.

Computing device 800 may further include a network interface device 808 which may communicate with a network 820. The computing device 800 also may include a video display unit 810 (e.g., a liquid crystal display (LCD) or a cathode ray tube (CRT)), an alphanumeric input device 812 (e.g., a keyboard), a cursor control device 814 (e.g., a mouse) and an acoustic signal generation device 816 (e.g., a speaker). In one embodiment, video display unit 810, alphanumeric input device 812, and cursor control device 814 may be combined into a single component or device (e.g., an LCD touch screen).

Data storage device 818 may include a computer-readable storage medium 828 on which may be stored one or more sets of instructions 825 that may include instructions for a caching component, e.g., caching component 225, for carrying out the operations described herein, in accordance with one or more aspects of the present disclosure. Instructions 825 may also reside, completely or at least partially, within main memory 804 and/or within processing device 802 during execution thereof by computing device 800, main memory 804 and processing device 802 also constituting computer-readable media. The instructions 825 may further be transmitted or received over a network 820 via network interface device 808.

While computer-readable storage medium 828 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

Unless stated otherwise as apparent from the foregoing discussion, it will be appreciated that terms such as “receiving,” “positioning,” “performing,” “emitting,” “causing,” or the like may refer to the actions and processes of a computer system, or similar electronic computing device, that manipulates and transforms data represented as physical (e.g., electronic) quantities within the computer system's registers and memories into other data similarly represented as physical within the computer system memories or registers or other such information storage or display devices. Implementations of the methods described herein may be implemented using computer software. If written in a programming language conforming to a recognized standard, sequences of instructions designed to implement the methods can be compiled for execution on a variety of hardware platforms and for interface to a variety of operating systems. In addition, implementations of the present disclosure are not described with reference to any particular programming language. It will be appreciated that a variety of programming languages may be used to implement implementations of the present disclosure.

It should be noted that the methods and apparatus described herein are not limited to use only with medical diagnostic imaging and treatment. In alternative implementations, the methods and apparatus herein may be used in applications outside of the medical technology field, such as industrial imaging and non-destructive testing of materials. In such applications, for example, “treatment” may refer generally to the effectuation of an operation controlled by the treatment planning system, such as the application of a beam (e.g., radiation, acoustic, etc.) and “target” may refer to a non-anatomical object or area.

In the foregoing specification, the disclosure has been described with reference to specific exemplary implementations thereof. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense. Reference throughout this specification to “one embodiment” or “an embodiment” means that a particular feature, structure, or characteristic described in connection with the embodiments included in at least one embodiment. Thus, the appearances of the phrase “in one embodiment” or “in an embodiment” in various places throughout this specification are not necessarily all referring to the same embodiment.

The above description of illustrated implementations of the invention, including what is described in the Abstract, is not intended to be exhaustive or to limit the invention to the precise forms disclosed. While specific implementations of, and examples for, the invention are described herein for illustrative purposes, various equivalent modifications are possible within the scope of the invention, as those skilled in the relevant art will recognize. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such. Furthermore, the terms “first,” “second,” “third,” “fourth,” etc. as used herein are meant as labels to distinguish among different elements and may not necessarily have an ordinal meaning according to their numerical designation.

What is claimed is:

1. A method comprising:
dividing an image viewing space of a client device into a plurality of three-dimensional (3D) volumes, wherein the image viewing space presents a two-dimensional (2D) viewing plane of data;
determining, by a processing device, a set of 3D volumes of the image viewing space intersecting the 2D viewing plane;
retrieving, by the processing device from a data server, image data associated with each of the 3D volumes intersecting the 2D viewing plane; and
presenting a portion of the retrieved image data corresponding to a position of the 2D viewing plane within the image viewing space.

2. The method of claim 1, wherein each 3D volume of the image viewing space is mapped to image data corresponding to a same position of the 3D volume within the image viewing space.

3. The method of claim 1, wherein the image data comprising time series data of a plurality of images collected over time.

4. The method of claim 3, further comprising:
displaying a first subset of the time series data corresponding to a first time and the position of the 2D viewing pane within the image viewing space;
receiving, by the client device, an indication of a second time; and
displaying a second subset of the time series data of the plurality of images corresponding to the second time and the position of the 2D viewing plane within the image viewing space.

5. The method of claim 1, further comprising:
initiating retrieval of higher-resolution image data for the 3D volumes;
retrieving lower-resolution image data for the 3D volumes; and
displaying the lower-resolution image data until the higher-resolution image data has been retrieved.

6. The method of claim 1, wherein the image data comprises one or more of computed tomography (CT) data, magnetic resonance (MR) data, positron emission tomography (PET) data, and ultrasound data.

7. The method of claim 6, wherein presenting the portion of the retrieved image data comprises presenting a combination of two or more of the CT data, MR data, PET data, and ultrasound data.

8. The method of claim 1, further comprising:
receiving a second position of the 2D viewing plane within the image viewing space;
determining a second set of 3D volumes intersecting the 2D viewing plane at the second position;
retrieving image data corresponding to the second set of 3D volumes; and
displaying a portion of the image data for the second position of the 2D viewing plane.

9. The method of claim 1, further comprising:
determining a second set of 3D volumes of the image viewing space corresponding to image data with a high probability of being view; and
retrieving, from the data server, the image data corresponding to the second set of 3D volumes.

10. A client device comprising:
a memory; and
a processing device, operatively coupled to the memory, to:
divide an image viewing space of the client device into a plurality of three-dimensional (3D) volumes, wherein the image viewing space presents a two-dimensional (2D) viewing plane of data;
determine a set of 3D volumes of the image viewing space intersecting the 2D viewing plane;
retrieve, from a data server, image data associated with each of the 3D volumes intersecting the 2D viewing plane; and
present a portion of the retrieved image data corresponding to a position of the 2D viewing plane within the image viewing space.

11. The client device of claim 10, wherein each 3D volume of the image viewing space is mapped to image data corresponding to a same position of the 3D volume within the image viewing space.

12. The client device of claim 10, wherein the image data comprising time series data of a plurality of images collected over time.

13. The client device of claim 12, wherein the processing device is further to:
display, by the client device, a first subset of the time series data corresponding to a first time and the position of the 2D viewing plane within the image viewing space;
receive an indication of a second time; and
display a second subset of the time series data of the plurality of images corresponding to the second time and the position of the 2D viewing plane within the image viewing space.

14. The client device of claim 10, wherein the processing device is further to:
initiate retrieval of higher-resolution image data for the 3D volumes;
retrieve lower-resolution image data for the 3D volumes; and
display the lower-resolution image data until the higher-resolution image data has been retrieved.

15. The client device of claim 10, wherein the image data comprises one or more of computed tomography (CT) data, magnetic resonance (MR) data, positron emission tomography (PET) data, and ultrasound data.

16. The client device of claim 15, wherein presenting the portion of the retrieved image data comprises presenting a combination of two or more of the CT data, MR data, PET data, and ultrasound data.

17. The client device of claim 10, wherein the processing device is further to:
receive a second position of the 2D viewing plane within the image viewing space;
determine a second set of 3D volumes intersecting the 2D viewing plane at the second position;
retrieve image data corresponding to the second set of 3D volumes; and
display a portion of the image data for the second position of the 2D viewing plane.

18. The client device of claim 10, wherein the processing device is further to:
determine a second set of 3D volumes of the image viewing space corresponding to image data with a high probability of being view; and
retrieve, from the data server, the image data corresponding to the second set of 3D volumes.

19. A non-transitory computer-readable storage medium including instructions that, when executed by a processing device, cause the processing device to:
divide an image viewing space of a client device into a plurality of three-dimensional (3D) volumes, wherein the image viewing space presents a two-dimensional (2D) viewing plane of data;
determine, by the processing device, a set of 3D volumes of the image viewing space intersecting the 2D viewing plane;
retrieve, by the processing device from a data server, image data associated with each of the 3D volumes intersecting the 2D viewing plane; and
present a portion of the retrieved image data corresponding to a position of the 2D viewing plane within the image viewing space.

20. The non-transitory computer-readable storage medium of claim 19, wherein each 3D volume of the image viewing space is mapped to image data corresponding to a same position of the 3D volume within the image viewing space.

21. The non-transitory computer-readable storage medium of claim 19, wherein the image data comprising time series data of a plurality of images collected over time.

22. The non-transitory computer-readable storage medium of claim 21, wherein the processing device is further to:
display a first subset of the time series data corresponding to a first time and the position of the 2D viewing pane within the image viewing space;
receive an indication of a second time; and
display a second subset of the time series data of the plurality of images corresponding to the second time and the position of the 2D viewing plane within the image viewing space.

23. The non-transitory computer-readable storage medium of claim 19, wherein the processing device is further to:
initiate retrieval of higher-resolution image data for the 3D volumes;
retrieve lower-resolution image data for the 3D volumes; and
display the lower-resolution image data until the higher-resolution image data has been retrieved.

24. The non-transitory computer-readable storage medium of claim 19, wherein the image data comprises one or more of computed tomography (CT) data, magnetic resonance (MR) data, positron emission tomography (PET) data, and ultrasound data.

25. The non-transitory computer-readable storage medium of claim 24, wherein presenting the portion of the retrieved image data comprises presenting a combination of two or more of the CT data, MR data, PET data, and ultrasound data.

26. The non-transitory computer-readable storage medium of claim 19, wherein the processing device is further to:
receive a second position of the 2D viewing plane within the image viewing space;
determine a second set of 3D volumes intersecting the 2D viewing plane at the second position;
retrieve image data corresponding to the second set of 3D volumes; and
display a portion of the image data for the second position of the 2D viewing plane.

27. The non-transitory computer-readable storage medium of claim 19, wherein the processing device is further to:
determine a second set of 3D volumes of the image viewing space corresponding to image data with a high probability of being view; and
retrieve, from the data server, the image data corresponding to the second set of 3D volumes.
